(19) United States
(12) Patent Application Publication
(10) Pub. No.: US 2017/0065206 A1
(43) Pub. Date: Mar. 9, 2017

(54) DEVICE FOR DETERMINING A SPECIFIC POSITION OF A CATHETER

(71) Applicant: KONINKLIJKE PHILIPS N.V., EINDHOVEN (NL)

(72) Inventors: MELIKE BOZKAYA, EINDHOVEN (NL); ALBERTO FAZZI, EINDHOVEN (NL)

(21) Appl. No.: 15/306,590

(22) PCT Filed: Apr. 16, 2015
(86) PCT No.: PCT/EP2015/058218

§ 371 Date: Oct. 25, 2016

(30) Foreign Application Priority Data
Apr. 29, 2014 (EP) 14166325.2

(51) Int. Cl. A61B 5/06; A61B 5/00; A61B 6/00

(52) U.S. Cl. CPC A61B 5/065; A61B 6/504; A61B 5/6852; A61B 6/5211

(57) ABSTRACT

The present invention relates to a device (1) and a system for determining a specific position of a distal end (11) of a catheter (10) in an anatomical structure, a method for determining a specific position of a distal end (11) of a catheter (10) in an anatomical structure, a computer program element for controlling such device (1) and a computer readable medium having stored such computer program element. The device (1) comprises a catheter (10) with a distal end (11) and a position sensor (12) arranged spaced apart from the distal end (11) in a proximal direction. The position sensor (12) is configured to provide position data. The device (1) further comprises a processing unit (20), wherein the processing unit (20) is configured to process given anatomical data of the anatomical structure, to detect a path of the catheter (10) through the anatomical structure based on the anatomical data and the position data of the position sensor (12), and to determine a specific position of the distal end (11) of the catheter (10)in the anatomical structure based on the path and the position data of the position sensor (12).

### FIELD OF THE INVENTION

The present invention relates to a device and a system for determining a specific position of a distal end of a catheter in an anatomical structure, a method for determining a specific position of a distal end of a catheter in an anatomical structure, a computer program element for controlling such device and a computer readable medium having stored such computer program element.

### BACKGROUND OF THE INVENTION

For the diagnosis and intervention of e.g. Coronary Artery Diseases (CAD), the use of different medical modalities is known. X-ray, as one of the common modalities, is in use for the diagnosis of CAD and for guidance in interventional procedures. Thereby, X-ray images may provide silhouettes of anatomical structures as e.g. a vessel lumen and of interventional tools. Intravascular technologies, like intravascular ultrasound (IVUS), optical coherence tomography (OCT), fractional flow reserve (FFR), near-infrared spectroscopy (NIRS) and others, as further common modalities, may be used in order to gather more information about the internal structure and function of the vessel and e.g. plaque/tissue characteristics.

WO 2009/044321 A2 discloses a method for automatic detection and tracking of interventional tools. It comprises calculating differences between co-registered X-ray images and 2D projected image data of a preoperatively acquired 3D voxel volume and using these differences for showing the interventional tools. However, a detection and tracking of interventional tools, and in particular catheters, can still be improved.

### SUMMARY OF THE INVENTION

Hence, there may be need to provide a device, which allows a precise and easy determination of a specific position of a distal end of a catheter in an anatomical structure.

The object of the present invention is solved by the subject-matters of the independent claims, wherein further embodiments are incorporated in the dependent claims. It should be noted that the following described aspects of the invention apply also for the device and the system for determining a specific position of a distal end of a catheter in an anatomical structure, the method for determining a specific position of a distal end of a catheter in an anatomical structure, the computer program element and the computer readable medium.

According to the present invention, a device for determining a specific position of a distal end of a catheter in an anatomical structure is presented. The catheter may also be a guidewire or any other kind of interventional tool. The anatomical structure constrains the movement of the catheter and may be a vessel.

The device for determining a specific position of a distal end of a catheter comprises a catheter with a distal end and a position sensor arranged spaced apart from the distal end in a proximal direction. The position sensor may be an electromagnetic sensor. The position sensor is configured to provide position data. The position sensor may be integrated in the catheter, preferably not at the distal end of the catheter, but more proximally, so that the catheter will maintain his natural floppiness at the tip.

The device for determining a specific position of a distal end of a catheter further comprises a processing unit. The processing unit is configured to process given anatomical data of the anatomical structure. The given anatomical data describe the anatomical structure and may be e.g. the anatomy of a vessel tree. It may be provided by an anatomical data unit, as e.g. an X-ray unit, an angiography unit or the like.

The processing unit is further configured to detect a path of the catheter through the anatomical structure based on the anatomical data and the position data of the position sensor, and to determine a specific position of the distal end of the catheter in the anatomical structure based on the path and the position data of the position sensor. The specific position of the distal end of the catheter in the anatomical structure can also be detected by a user input based on the path and the position data of the position sensor. The path may be e.g. a pullback or pushforward path of the catheter in a vessel. It can be a path in which a catheter is moved in a vessel to obtain e.g. intravascular data.

As a result, a device for determining a specific position of a distal end of a catheter in an anatomical structure is provided. To determine a specific position of the distal end of the catheter, the exact distance of the position sensor to the distal end of the catheter has to be known. Therefore, the position of the position sensor may comprise the position data in the anatomical structure and the position on the catheter.

The device allows a precise and easy determination of the specific position of the distal end of the catheter in the anatomical structure. This can be achieved, while the position sensor is arranged spaced apart from the distal end in a proximal direction and need not to be arranged at the catheter tip. This allows maintaining a certain flexibility of the catheter tip and avoids injuries of the anatomical structure.

The invention can be applied e.g. to coronary procedures and other field applications in which a path shall be or can be identified.

In an example, the device for determining a specific position of a distal end of a catheter in an anatomical structure further comprises an intravascular data acquisition sensor arranged essentially at the distal end of the catheter and configured to provide intravascular data. The intravascular data acquisition sensor may be based on intravascular technologies, like intravascular ultrasound (IVUS), optical coherence tomography (OCT), fractional flow reserve (FFR), near-infrared spectroscopy (NIRS) and the like. The intravascular data acquisition sensor may also be an intravascular imaging sensor.

The processing unit may be configured to register intravascular data with position data of the position sensor and/or with the anatomical data based on the position data of the position sensor.

Exemplarily and in other words, by means of the electromagnetic position sensor, a electromagnetic based position localization of the intravascular data acquisition sensor and of intravascularly acquired data within the coronary artery tree is provided. The position of the intravascular data acquisition sensor is determined based on a predefined distance between the intravascular data acquisition sensor and the position sensor. Since the position sensor and the intravascular data acquisition sensor are not collocated and not rigidly linked, the position of the intravascular data acquisition sensor is inferred based on the location of the position sensor and the prior knowledge of the anatomy of the vessel tree. Thereby, a catheter tracking and registration of related intravascularly acquired data to the anatomy without the continuous use of harmful X-ray radiation is allowed.

The position data may be used to extend the detection of the path beyond the given anatomical data. Exemplarily and in other words, it is possible that e.g. during pullback the position sensor exits the given anatomical data, e.g. the initial X-ray field of view, and therefore exits the recognized pullback path. If so, the electromagnetic tracked locations of the position sensor can be used during pullback to extend the topology of the pullback path outside the initial X-ray field of view.

In an example, the device for determining a specific position of a distal end of a catheter in an anatomical structure further comprises a feedback unit configured to provide feedback about the movement of the catheter relative to the path. The feedback is preferably based on the position data. Exemplarily and in other words, the system can give feedback and guidance about correctness of e.g. the pullback process depending on the intravascular system. Preferably, there can be limitations on the speed of the pullback and/or any other aspect of the intravascular system or catheter. Based on the position-tracking by the position sensor, e.g. feedback, user warnings and visual information can be given to the user. In particular, if the pullback is performed manually, a warning could be raised if the pullback speed would be too high with respect to the patient or the characteristics of the intravascular data acquisition sensor.

In a further example, the device for determining a specific position of a distal end of a catheter in an anatomical structure further comprises a display unit configured to present a synchronized and/or registered view of the intravascular data, the position data and/or the anatomical data based on the position data. In other words, the anatomical location at which the intravascular data was acquired is now known. Preferably, the position sensor and/or the intravascular data acquisition sensor are visible in the anatomical data. The display unit can also be configured to present the feedback and guidance of the feedback unit.

In a further example, the path is defined by a start point, an end point or both, which is/are identified either by user input e.g. by means of the display unit or by the positions of the data acquisition sensor and/or the position sensor.

According to the present invention, also a system for determining a specific position of a distal end of a catheter in an anatomical structure is presented. It comprises an anatomical data unit, a catheter comprising a distal end and a position sensor arranged spaced apart from the distal end in a proximal direction, and a processing unit.

The anatomical data unit is configured to provide anatomical data. The anatomical data unit may be an X-ray unit, an angiography unit or the like.

The position sensor is configured to provide position data. The position sensor may be an electromagnetic sensor.

The processing unit is configured to detect a path of the catheter through an anatomical structure based on the anatomical data and the position data of the position sensor. The processing unit is further configured to determine a specific position of the distal end of the catheter in the anatomical structure based on the path and the position data of the position sensor.

According to the present invention, also a method for determining a specific position of a distal end of a catheter in an anatomical structure is presented. In an example, the method comprises the following steps, not necessarily in this order:

a) providing a catheter comprising a distal end and an position sensor arranged spaced apart from the distal end in a proximal direction,

b) providing position data of the position sensor,

c) detecting a path of the catheter through an anatomical structure based on the position data of the position sensor and given anatomical data, and

d) determining a specific position of the distal end of the catheter in the anatomical structure based on the path and the position data of the position sensor.

An exemplarily different order of the method steps is shown in the following description of the Figures.

In a further example, the method comprises the additional following steps, not necessarily in this order:
providing an anatomical data unit,
registering the position sensor with the anatomical data unit,
generating anatomical data, and
providing the anatomical data.

The step of registering the position sensor with the anatomical data unit concerns a coordinate system registration.

In a further example, the method comprises the additional following steps, not necessarily in this order:
providing an intravascular data acquisition sensor arranged essentially at the distal end of the catheter and configured to provide intravascular data, and
registering intravascular data with position data of the position sensor and/or with the anatomical data based on the position data of the position sensor.

The method for determining a specific position of a distal end of a catheter in an anatomical structure may also comprise the following steps, not necessarily in this order:

a) providing image data showing a catheter comprising a proximal part, a distal part and a position sensor arranged between both parts at the end of the proximal part,

b) providing position data of the position sensor in the catheter in an anatomical structure,

c) providing anatomical data,

d) detecting a path of the proximal part of the catheter through the anatomical structure based on the position data of the position sensor and the anatomical data, and

e) determining a specific position of the distal end of the catheter in the anatomical structure based on the path and the position data of the position sensor.

The path of the proximal part of the catheter through the anatomical structure can also be detected based on user input e.g. by means of a display unit.

In a further example of the present invention, a computer program element for controlling such device is presented, which, when being executed by a processing device, is adapted to perform the method steps shown above.

In a further example of the present invention, a computer readable medium is presented, which has stored above program element.

It shall be understood that the device and the system for determining a specific position of a distal end of a catheter in an anatomical structure, the method for determining a specific position of a distal end of a catheter in an anatomical structure, the computer program element and the computer readable medium according to the independent claims have similar and/or identical preferred embodiments, in particular, as defined in the dependent claims. It shall be understood further that a preferred embodiment of the invention can also be any combination of the dependent claims with the respective independent claim.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the accompanying drawings:

FIG. 1 shows schematically and exemplarily an embodiment of a device and a system for determining a specific position of a distal end of a catheter in an anatomical structure.

FIG. 2 shows schematically and exemplarily an embodiment of a method for determining a specific position of the distal end of the catheter in a vessel.

FIG. 3 shows the embodiment of the method according to FIG. 2 in a different illustration.

FIG. 4 shows schematically and exemplarily a further embodiment of a method for determining a specific position of the distal end of the catheter in an anatomical structure.

### DETAILED DESCRIPTION OF EMBODIMENTS

While intravascular imaging technologies provide high resolution assessment of the plaque and vessel walls, the generated images do not allow determination of the location and orientation of a specific intravascular image frame in 3D space. In order to solve the localization problem, different co-registration techniques have been proposed.

The intravascular imaging modalities provide information on the local characteristics of the vessels. However, they cannot provide information about the global position of the imaged location. To link the local data with the larger vessel geometry, fluoroscopic X-ray data is used. By detecting and tracking the tip of the intravascular imaging device in the X-ray images, the intravascular imaging data can be registered to the vessel geometry.

However, the process is not straightforward. Image-based detection of the device tip may be erroneous. Furthermore, the frame rate of the X-ray system and the intravascular data sensor may differ, requiring interpolation of the position. Finally, radiation emission during fluoroscopy is not desirable both for the patient and for the operator.

Alternatively, 3D position information can be obtained by an electromagnetic (EM) tracking technology. EM sensor coils can be used in medical interventions to assist navigation. The coils are typically located at the device distal end as this constitutes the part of the device that is typically more important to track. However, the size and stiffness of typical coil sensors often exclude their applicability to devices meant to navigate the coronary arteries during PCIs as a catheter or a guidewire with a stiff tip could cause damage to the vessels.

According to an exemplary embodiment of this invention, an EM sensor coil is placed proximally (e.g. in the part of the device that would be close to coronary ostium in a coronary intervention), its location is tracked during the pullbacks performed for the acquisition of IVUS data from a probe typically placed at the distal end of the device, and the EM sensor coordinates are converted to IVUS probe coordinates based on the known pullback path of the device. Thereby, the trajectory of the intravascular probe can be used to register the intravascular data with the vessel tree.

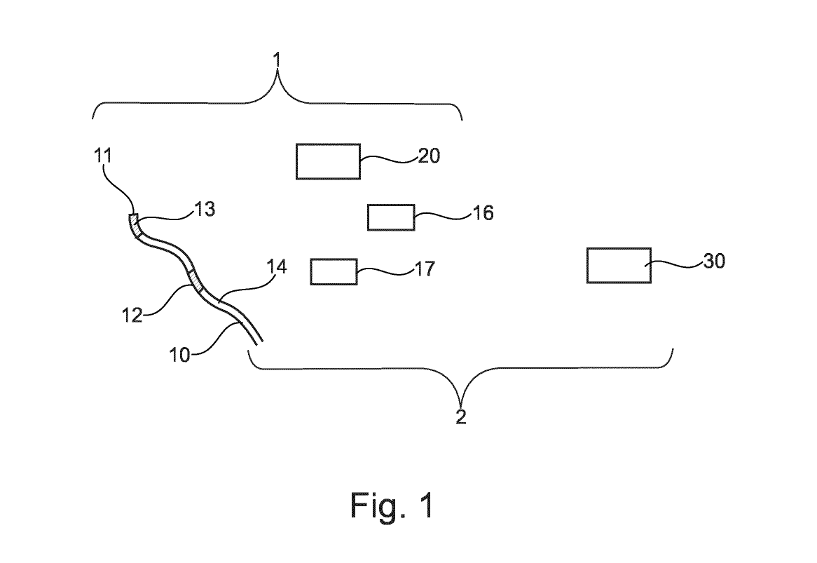

FIG. 1 shows schematically and exemplarily an embodiment of a device 1 and a system 2 for determining a specific position of a distal end 11 of a catheter 10 in an anatomical structure (not shown). The anatomical structure may be a vessel.

The device 1 comprises a catheter 10 with a distal end 11 and a position sensor 12 arranged spaced apart from the distal end 11 in a proximal part 14. The position sensor 12 is here an electromagnetic sensor 12. The position sensor 12 is configured to provide position data. The position sensor 12 is integrated in the catheter, not at the distal end 11 of the catheter 10, but more proximally, so that the catheter 10 will maintain the required floppiness and the required small diameter at the tip.

The system 2 further comprises an electromagnetic (EM) tracking unit (not shown) giving the position information of the position sensor to a processing unit 20 of the device 1.

The processing unit 20 is configured to process given anatomical data of the anatomical structure, e.g. the anatomy of a vessel tree. The given anatomical data are provided by an anatomical data unit 30, as e.g. an X-ray unit, an angiography unit or the like.

The processing unit 20 is further configured to detect a path of the catheter 10 through the anatomical structure based on the anatomical data and the position data of the position sensor 12, and to determine a specific position of the distal end 11 of the catheter 10 in the anatomical structure based on the path, on the position data of the position sensor 12 and on the a priory knowledge of the device properties (e.g. of the linear distance between distal tip and position sensor).

The path may be e.g. a pullback or pushforward path of the catheter in a vessel, while pullback is preferred.

As a result, a device 1 for determining a specific position of a distal end 11 of a catheter 10 in an anatomical structure is provided. It allows a precise and easy determination of the specific position of the distal end 11 of the catheter 10 in the anatomical structure. This can be achieved, while the position sensor 12 is arranged spaced apart from the distal end 11 in a proximal direction and need not to be arranged at the catheter tip. This allows maintaining a certain flexibility and a limited diameter of the catheter tip and avoids injuries of the anatomical structure.

The device 1 further comprises an intravascular data acquisition sensor 13 arranged essentially at the distal end 11 of the catheter 10 and configured to provide intravascular data. The intravascular data acquisition sensor is here an intravascular ultrasound (IVUS) probe. The processing unit 20 is configured to register intravascular data with position data of the electromagnetic position sensor 12 and/or with the anatomical data based on the position data.

As a result, electromagnetic based position localization of the intravascular data acquisition sensor 13 and of intravascularly acquired data within e.g. a coronary artery tree are provided. The position of the intravascular data acquisition sensor 13 is determined based on a predefined distance between the intravascular data acquisition sensor 13 and the electromagnetic position sensor 12. Since the position sensor 12 and the intravascular data acquisition sensor 13 are linked by the structure of the catheter 10, the position of the intravascular data acquisition sensor 13 is inferred based on the location of the position sensor 12 and the prior knowledge of the anatomy of the vessel tree. Thereby, a catheter tracking and registration of related intravascularly acquired data to the anatomy without the continuous use of harmful X-ray radiation is made possible.

The device 1 further comprises a feedback unit 16 to provide feedback about the movement of the catheter 10 relative to the path. The feedback is based on the position data. The system can give feedback and guidance about correctness of e.g. the pullback process depending on the intravascular system. There can be limitations on the speed of the pullback and/or any other aspect of the catheter 10. Based on the position-tracking by the position sensor 12, e.g. feedback, user warnings and visual information can be given to the user. In particular, if the pullback is performed manually, a warning could be raised if the pullback speed would be too high with respect to the patient or the characteristics of the intravascular data acquisition sensor 13.

The device 1 further comprises a display unit 17 to present a synchronized view of the intravascular data, the position data and/or the anatomical data based on the position data. The position sensor 12 and/or the intravascular data acquisition sensor 13 are visible in the anatomical data. The display unit 17 also presents the feedback and guidance of the feedback unit 16. The feedback unit 16 provides feedback about the movement of the catheter 10 relative to the path. The feedback unit 16 can give feedback and guidance about correctness of e.g. the pullback process depending on the intravascular system. There can be limitations on the speed of the pullback and/or any other aspect of the intravascular system or catheter 10. Based on the position-tracking by the position sensor 12, e.g. feedback, user warnings and visual information can be given to the user. In particular, if the pullback is performed manually, a warning could be raised if the pullback speed would be too high with respect to the patient or the characteristics of the intravascular data acquisition sensor 13.

The system 2 for determining a specific position of a distal end 11 of a catheter 10 in an anatomical structure comprises the processing unit 20, the above described catheter 10, and additionally, an anatomical data unit 30. The anatomical data unit 30 provides anatomical data describing the anatomical structure, as e.g. the anatomy of a vessel tree. The anatomical data unit 30 may be e.g. an X-ray unit, an angiography unit or the like.

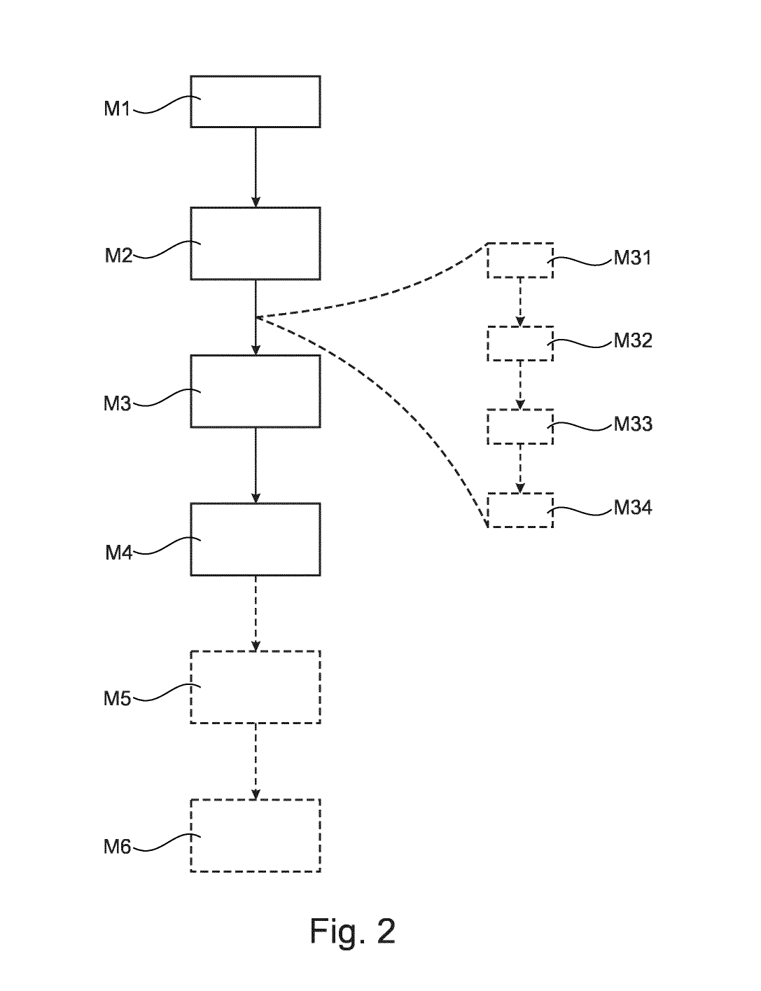

FIG. 2 shows schematically and exemplarily an embodiment of a method for determining a specific position of the distal end 11 of the catheter 10 in a vessel. The method comprises the following steps not necessarily in this order:

M1 providing the catheter 10 comprising the distal end 11 and the position sensor 12 arranged spaced apart from the distal end 11 in a proximal direction,

M2 providing position data of the position sensor 12,

M3 detecting a path of the catheter 10 through a vessel based on the position data of the position sensor 12 and given anatomical data,

M4 determining a specific position of the distal end 11 of the catheter 10 in the vessel based on the path and the position data of the position sensor 12,

M5 providing the intravascular data acquisition sensor 13 arranged essentially at the distal end 11 of the catheter 10 and configured to provide intravascular data, and

M6 registering intravascular data with position data of the position sensor 12 and/or with the anatomical data based on the position data of the position sensor 12.

The path of the catheter through the vessel can also be detected based on user input. As stated above, the shown order of method steps is not mandatory and does not reflect a temporal course. For example, the intravascular data acquisition sensor 13 of step M5 is attached to the catheter 10 provided in step M1. The provision of intravascular data would start with step M6 and occurs continuously during the pullback.

Concerning the given anatomical data, the method comprises the further optional steps of:

M31 providing the anatomical data unit 30,

M32 registering the position sensor 12 with the anatomical data unit 30,

M33 generating anatomical data, and

M34 providing the anatomical data.

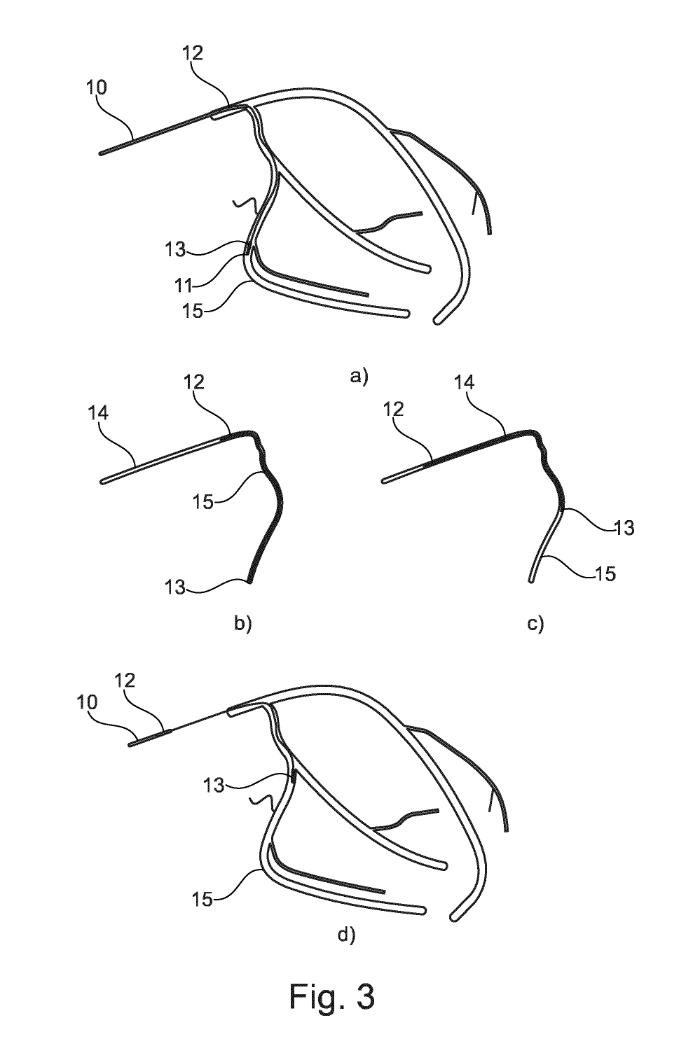

The method is further described in the following with reference to FIGS. 2 and 3. FIG. 3 shows the embodiment of the method according to FIG. 2 in a different illustration. In FIG. 3a, the catheter 10 is shown in a vessel 15 of a vessel tree, as presented e.g. by an X-ray image. The catheter comprises a distal end 11, a position sensor 12 and an intravascular data acquisition sensor 13. The intravascular data acquisition sensor 13 is shown at its initial location. The position sensor 12 will be tracked as explained in the following.

According to method step Ml, the catheter 10 is provided. The catheter 10 has an electromagnetic (EM) coil sensor 12 as position sensor 12. The EM sensor 12 provides position data according to method step M2.

According to optional method step M5, the catheter 10 is provided with the intravascular data acquisition sensor 13 arranged essentially at the distal end 11 of the catheter 10 and configured to provide intravascular data. The intravascular data acquisition sensor 13 is exemplarily an IVUS probe 13 located at the catheter's tip with a certain distance between the IVUS probe 13 and the EM sensor 12 located more proximal. The distance is the linear distance along the catheter and depends on the catheter construction/geometry and it is known a priori.

The proximal EM sensor 12 is placed in such a way that it would never be located further than the coronary ostia (e.g. in ascending aorta) while the IVUS sensor 13 is as deep as needed in the coronary tree. The IVUS probe 13 provides intravascular data. This is to be understood as an example only. The catheter 10 is designed so that the EM sensor 12 would not have to enter the small vessels that should be imaged by means of the IVUS sensor 13 but that could be damaged by the rigidity or the thickness of the EM sensor 12.

As a result, the catheter provides 3D location information during intravascular imaging.

In FIG. 3b, a pullback path 14 of the catheter 10 through the vessel 15 is shown. The location of the EM sensor 12 is tracked and the location of the IVUS sensor 13 is inferred as explained in the following.

According to method step M3, the path of the catheter 10 through the vessel 15 is detected or identified based on the position data of the position sensor 12 and given anatomical data. The detection of the path requires either the user to manually indicate the tip of the catheter or requires an algorithm to automatically detect the tip based on image processing. The path of the catheter through the vessel can also be detected in that the user clicks and thereby indicates start and end of the pullback path in the anatomical data, and the in-between vessel is then detected as the pullback path. Here, as anatomical data, a coronary artery tree is detected from contrasted angiography. The path is a pullback path of the catheter 10 through the vessel 15. The pullback path is detected based on the vessel tree information (roadmap) as given anatomical data and the catheter location as position data of the position sensor 12.

The anatomical data is e.g. “given” in that the anatomical data unit 30 is provided (step M31), the position sensor 12 is registered with the anatomical data unit 30 (step M32), anatomical data are generated (step M33), and anatomical data are provided (step M34).

In FIG. 3c, the catheter 10 is pulled back through the vessel 15 on the pullback path 14. The location of the EM sensor 12 is tracked and the location of the IVUS sensor 13 is inferred. The location is inferred by knowing the location of the position sensor 12, knowing the pullback path and matching the linear distance between the position sensor 12 and the catheter's tip to the 3D shape of the pullback path. In FIG. 3d, the catheter 10 is shown in a pulled back position. The position of the EM sensor 12 is again or still tracked and the position of the IVUS sensor 13 is inferred or estimated. The present position of the IVUS sensor 13 is shown analogue to FIG. 3a in an X-ray image.

According to method step M4, the specific position of the distal end 11 of the catheter 10 in the vessel is determined based on the path and the position data of the position sensor 12. Here, preferably 3D position information obtained from the EM-sensor 12 is used to locate the intravascular imaging sensor 13 on the vessel tree along the previously identified pullback path 14.

As also shown, the position data may be used to extend the detection of the path beyond the given anatomical data. During pullback, the position sensor exits the given anatomical data, here the initial X-ray field of view, and therefore exits the recognized pullback path. Then, the electromagnetic tracked locations of the position sensor can be used during pullback to extend the topology of the pullback path outside the initial X-ray field of view. This is based on the assumption that the path of the catheter 10 outside the known anatomy is substantially equivalent to the trajectory of the EM position sensor 12.

As stated above, according to optional step M5, the intravascular data acquisition sensor 13 is arranged at the distal end 11 of the catheter 10 and configured to provide intravascular data. According to optional step M6, the intravascular data are registered with position data of the position sensor 12 based on the position data of the position sensor 12. In other words, the position or location information and the acquired intravascular data are merged. It is also possible to register the intravascular data with the anatomical data based on the position data of the position sensor 12.

In the following, the steps of the method for determining a specific position of the distal end 11 of the catheter 10 are explained again, but in a more detailed embodiment:

The position sensor 12 is registered with the anatomical data unit 30, or in other words, the coordinate system of the EM sensing unit is registered to the coordinate system of the anatomical data and/or the coordinate system of the X-ray system 30.

The catheter 10 with the intravascular data acquisition sensor 13 is advanced to the distal position of e.g. a lesion in a coronary artery for intravascular data acquisition.

Anatomical data are generated by e.g. using a contrast agent and high dose exposure images, such that an angiogram is generated in which the coronary arteries can be recognized. This angiogram image is used to identify the pullback path 14. Afterwards, it can be used during the pullback to show the tracked locations of the intravascular sensor 13, and thus providing visual feedback on the pullback process. The angiogram image can be static (single frame) or dynamic in which the cardiac motion is visible.

In order to find the pullback path 14, both the IVUS probe 13 (proximal) and the EM sensor 12 (distal) are visible on the X-ray image. The pullback path identification can be done based on user input (user clicks the start and end of pullback vessel). Or it is possible to detect the IVUS probe 13 and the EM sensor 12 image-based or based on tracked coordinates of the EM sensor 12. In other words, if the X-ray image is an angiogram with contrast agent, then the sensors 12 and 13 are not visible and the user needs to click to identify. On the other hand, if there is an image-based detection, then there is an X-ray image with no contrast agent in addition to the angiogram, so that the sensors 12 and 13 are visible. Based on these locations, the pullback path 14 is identified.

The catheter pullback is performed (automatically or manually) while the EM-sensor 12 is being tracked. The EM sensor 12 movements are used to infer the position of the distal tip 11 based on the previously identified pullback path 14 and the prior knowledge of the catheter geometry. During the pullback, the tip of the catheter 10 can be visualized (with or without use of X-ray) and the 3D tracked locations can be stored. At the end of the pullback, the IVUS probe 13 can be once again detected (e.g. user input or probe detecting algorithm) in order to use the marked IVUS locations (start and end) in order to increase the accuracy of the transformation between EM coordinates and IVUS coordinates.

In order to show where IVUS images are generated on the coronary artery tree, a synchronized view of IVUS and X-ray can be created. For this, tracked and stored EM-locations should be correlated with the intravascular data (i.e. IVUS frames).

To visualize the inferred IVUS probe locations on the coronary artery, an indicator (i.e. marker) can be used. For this phase, means to visualize the intravascular data with respect to the coronary tree, means to scan through it, perform measurements, data analysis, and detection of clinically relevant features are provided. Further, time synchronization between anatomical data and position data is needed. In case the anatomical structure could move during the procedure, this movement should be properly taken into account. As examples, if the anatomical data is pre-acquired data and therefore static and not acquired in real time, the movement components of the position sensor that refer to movements of the anatomy rather than movements of the catheter should be filtered/compensated so that the position of the catheter could be properly relate to the static pre-acquired anatomy information. If the anatomical data is dynamic and/or live and therefore reflecting the anatomy movements, the time synchronization of position data and anatomical data should be sufficient to ensure correct interpretation of the data.

The invention can also be applied to any other intravascular data acquisition than IVUS acquisition. Besides to coronary procedures, the invention can also be applied to any other field application in which a path can be identified.

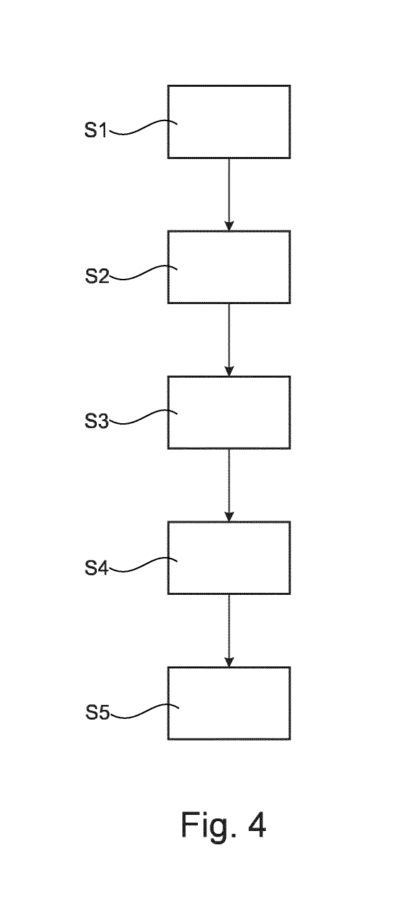

The invention can be used for image guided interventions where an intravascular technology is in-use. FIG. 4 shows schematically and exemplarily an embodiment of a method for determining a specific position of the distal end 11 of the catheter 10 in an anatomical structure. The method comprises the following steps not necessarily in this order:

S1 providing image data showing the catheter 10 comprising the proximal part 14, the distal part and the position sensor 12 arranged between both parts at the end of the proximal part 14,

S2 providing position data of the position sensor 12 in the catheter 10 in an anatomical structure,

S3 providing anatomical data,

S4 detecting a path of the proximal part 14 of the catheter 10 through the anatomical structure based on the position data of the position sensor 12 and the anatomical data, and

S5 determining a specific position of the distal end 11 of the catheter 10 in the anatomical structure based on the path and the position data of the position sensor 12.

The order on at least steps Si to S3 is arbitrary; they can e.g. also be effected simultaneously.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it, which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A device for determining a specific position of a distal end of a catheter in an anatomical structure, comprising
a catheter comprising a distal end and a position sensor arranged spaced apart from the distal end in a proximal direction, and
a processing unit,
wherein the position sensor is configured to provide position data, and
wherein the processing unit is configured
to process given anatomical data of the anatomical structure,
to detect a path of the catheter, through the anatomical structure based on the anatomical data and the position data of the position sensor, and
to determine a specific position of the distal end of the catheter in the anatomical structure based on the path and the position data of the position sensor.

2. Device according to claim 1, further comprising an intravascular data acquisition sensor arranged essentially at the distal end of the catheter and configured to provide intravascular data.

3. Device according to claim 2, wherein the processing unit is further configured to register intravascular data with position data of the position sensor and/or with the anatomical data based on the position data of the position sensor.

4. Device according to claim 1, wherein the position data are used to extend the detection of the path beyond the given anatomical data.

5. Device according to claim 1, further comprising a feedback unit configured to provide feedback about the movement of the catheter relative to the path, preferably based on the position data.

6. Device according to claim 1, wherein the path is defined by a start and/or an end point identified either by user input or by the positions of the data acquisition sensor and/or the position sensor

7. Device according to claim 1, wherein the device further comprises a display unit configured to present a synchronized view of the intravascular data and the anatomical data based on the position data.

8. A system for determining a specific position of a distal end of a catheter in an anatomical structure, comprising an anatomical data unit and a device according to claim 1, wherein the anatomical data unit is configured to provide said given anatomical data.

9. System according to claim 1, wherein the position sensor is an electromagnetic sensor and/or the anatomical data unit is an X-ray unit, preferably an angiography unit.

10. A method for determining a specific position of a distal end of a catheter in an anatomical structure, comprising the steps of
a) providing image data showing a catheter comprising a proximal part, a distal part and a position sensor arranged between both parts at the end of the proximal part,
b) providing position data of the position sensor in the catheter in an anatomical structure,
c) providing anatomical data,
d) detecting a path of the proximal part of the catheter through the anatomical structure based on the position data of the position sensor and the anatomical data, and
e) determining a specific position of the distal end of the catheter in the anatomical structure based on the path and the position data of the position sensor.

11. Method according to claim 10, comprising the steps of
a) providing a catheter comprising a distal end and a position sensor arranged spaced apart from the distal end in a proximal direction,
b) providing position data of the position sensor,
c) detecting a path of the catheter through an anatomical structure based on the position data of the position sensor and given anatomical data, and
d) determining a specific position of the distal end of the catheter in the anatomical structure based on the path and the position data of the position sensor.

12. Method according to claim 11, comprising the further steps of
providing an anatomical data unit,
registering the position sensor with the anatomical data unit,
generating anatomical data, and
providing the anatomical data.

13. Method according to claim 10, comprising the further steps of
providing an intravascular data acquisition sensor arranged essentially at the distal end of the catheter and configured to provide intravascular data, and registering intravascular data with position data of the position sensor and/or
with the anatomical data based on the position data of the position sensor.

14. A computer program element for controlling a device according to claim 1, which, when being executed by a processing device, is adapted to perform the method steps for determining a specific position of a distal end of a catheter in an anatomical structure.

15. A computer readable medium having stored the program element of claim 14.
